(19) United States
(12) Patent Application Publication
(10) Pub. No.: US 2025/0359729 A1
(43) Pub. Date: Nov. 27, 2025

(54) MEDICAL DEVICE, MEDICAL SYSTEM, LEARNING DEVICE, OPERATION METHOD OF MEDICAL DEVICE, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Nanako SATO, Sagamihara-shi (JP)

(73) Assignee: OLYMPUS MEDICAL SYSTEMS CORP., Tokyo (JP)

(21) Appl. No.: 19/290,582

(22) Filed: Aug. 5, 2025

Related U.S. Application Data
(63) Continuation of application No. PCT/JP2023/004401, filed on Feb. 9, 2023.

(51) Int. Cl. A61B 1/00; A61B 1/04; A61B 1/06

(52) U.S. Cl. CPC A61B 1/000094; A61B 1/000096; A61B 1/0005; A61B 1/043; A61B 1/0638

(57) ABSTRACT

A medical device includes a processor including hardware, the processor being configured to: acquire a thermally denatured image including at least a thermally denatured region; specify a specific region included in the thermally denatured image; set, based on the specific region, a detection range serving as a target range for detecting the thermally denatured region in the thermally denatured image; determine whether the thermally denatured region is included in the detection range; and output thermal denaturation information when the thermally denatured region is included in the detection range.

### CROSS REFERENCES TO RELATED APPLICATIONS

This application is a continuation of International Application No. PCT/JP2023/004401, filed on Feb. 9, 2023, the entire contents of which are incorporated herein by reference.

### BACKGROUND

### 1. Technical Field

The present disclosure relates to a medical device, a medical system, a learning device, an operation method of the medical device, and a computer-readable recording medium.

### 2. Related Art

In the related art, in the medical field, a technique for visualizing a cauterization state of a subject such as a biological tissue using an energy device or the like is known (refer to, for example, WO 2020/054723 A). In this technique, a subject is irradiated with excitation light, and an image and information including fluorescence image data generated based on an imaging signal acquired by capturing fluorescence generated from a thermally invasive region of the subject by receiving the excitation light are displayed, thereby visualizing a cauterization state for a user such as an operator.

### SUMMARY

In some embodiments, a medical device includes a processor including hardware, the processor being configured to: acquire a thermally denatured image including at least a thermally denatured region; specify a specific region included in the thermally denatured image; set, based on the specific region, a detection range serving as a target range for detecting the thermally denatured region in the thermally denatured image; determine whether the thermally denatured region is included in the detection range; and output thermal denaturation information when the thermally denatured region is included in the detection range.

In some embodiments, a learning device includes a processor including hardware, the processor being configured to generate a learned model by performing machine learning using teacher data in which a fluorescence image obtained by capturing fluorescence generated by irradiating a biological tissue with excitation light and a white light image obtained by capturing an image generated by irradiating the biological tissue with white light are used as input data, and information indicating a relationship between a thermally denatured region extracted from the fluorescence image and a specific region specified from the white light image is used as output data.

In some embodiments, a medical system includes: a light source device including a light source configured to emit excitation light for exciting an advanced glycation end-product generated by subjecting a biological tissue to a thermal treatment; an imaging device including an imaging element configured to generate an imaging signal by capturing fluorescence emitted by the excitation light; and a medical device including a processor including hardware, the processor being configured to: acquire a thermally denatured image including at least a thermally denatured region; specify a specific region included in the thermally denatured image; set, based on the specific region, a detection range serving as a target range for detecting the thermally denatured region in the thermally denatured image; determine whether the thermally denatured region is included in the detection range; and output thermal denaturation information when the thermally denatured region is included in the detection range.

In some embodiments, provided is an operation method of a medical device comprising a processor. The operation method causes the processor to execute: acquiring a thermally denatured image including at least a thermally denatured region; specifying a specific region included in the thermally denatured image; setting, based on the specific region, a detection range serving as a target range for detecting the thermally denatured region in the thermally denatured image; determining whether the thermally denatured region is included in the detection range; and outputting thermal denaturation information when the thermally denatured region is included in the detection range.

In some embodiments, provided is a non-transitory computer-readable recording medium with an executable program stored thereon. The program causes a processor of a medical device driven to execute: acquiring a thermally denatured image including at least a thermally denatured region; specifying a specific region included in the thermally denatured image; setting, based on the specific region, a detection range serving as a target range for detecting the thermally denatured region in the thermally denatured image; determining whether the thermally denatured region is included in the detection range; and outputting thermal denaturation information when the thermally denatured region is included in the detection range.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a schematic configuration of an endoscope system according to a first embodiment;

FIG. 2 is a block diagram illustrating a functional configuration of a main part of the endoscope system according to the first embodiment;

FIG. 3 is a diagram schematically illustrating wavelength characteristics of excitation light emitted by two light source units according to the first embodiment;

FIG. 4 is a diagram schematically illustrating a configuration of a pixel unit according to the first embodiment;

FIG. 5 is a diagram schematically illustrating a configuration of a color filter according to the first embodiment;

FIG. 6 is a diagram schematically illustrating sensitivity and a wavelength band of each filter according to the first embodiment;

FIG. 7A is a diagram schematically illustrating signal values of G pixels of an imaging element according to the first embodiment;

FIG. 7B is a diagram schematically illustrating signal values of R pixels of the imaging element according to the first embodiment;

FIG. 7C is a diagram schematically illustrating signal values of B pixels of the imaging element according to the first embodiment;

FIG. 8 is a diagram schematically illustrating a configuration of a cut filter according to the first embodiment;

FIG. 9 is a diagram schematically illustrating transmission characteristics of the cut filter according to the first embodiment;

FIG. 10 is a flowchart illustrating an outline of processing executed by the control device according to the first embodiment;

FIG. 11 is a diagram schematically illustrating a specific region in a white light image specified by a specification unit according to the first embodiment;

FIG. 12 is a diagram schematically illustrating a detection range set by a setting unit according to the first embodiment;

FIG. 13 is a diagram illustrating an example of a display image displayed by the display device according to the first embodiment;

FIG. 14 is a diagram illustrating an example of a display image displayed by the display device according to the first embodiment;

FIG. 15 is a diagram illustrating a schematic configuration of an endoscope system according to a second embodiment;

FIG. 16 is a block diagram illustrating a functional configuration of a medical device according to a second embodiment.

### DETAILED DESCRIPTION

Hereinafter, modes for carrying out the present disclosure will be described in detail with reference to the drawings. Note that the present disclosure is not limited to the following embodiments. In addition, each drawing referred to in the following description merely schematically illustrates a shape, a size, and a positional relationship to an extent that a content of the present disclosure can be understood. That is, the present disclosure is not limited only to the shape, the size, and the positional relationship illustrated in each drawing. Furthermore, in the description of the drawings, the same portions will be denoted by the same reference numerals. Furthermore, as an example of an endoscope system according to the present disclosure, an endoscope system including a rigid endoscope and a medical imaging device will be described.

### First Embodiment

### Configuration of Endoscope System

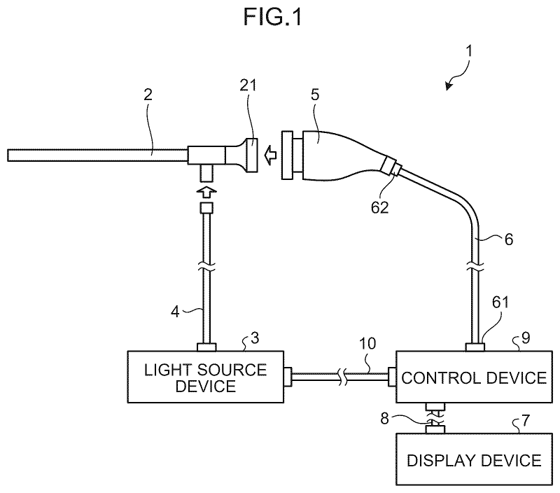

FIG. 1 is a diagram illustrating a schematic configuration of an endoscope system according to a first embodiment. An endoscope system 1 illustrated in FIG. 1 is a system that is used in a medical field and observes and treats a biological tissue in a subject such as a living body. Note that, in the first embodiment, a rigid endoscope system using a rigid endoscope (an insertion unit 2) illustrated in FIG. 1 will be described as the endoscope system 1, but the disclosure is not limited thereto, and for example, an endoscope system including a flexible endoscope may be used. Furthermore, the endoscope system 1 can also be applied to a medical microscope, a medical surgical robot system, or the like that includes a medical imaging device that captures a subject and performs surgery, processing, or the like while displaying an observation image based on an imaging signal (image data) captured by the medical imaging device on a display device. In addition, in recent years, in the medical field, minimally invasive treatment using an endoscope, a laparoscope, or the like has been widely performed. For example, as minimally invasive treatment using an endoscope, a laparoscope, or the like, endoscopic submucosal dissection (ESD), laparoscopy and endoscopy cooperative surgery (LECS), non-exposed endoscopic wall-inversion surgery (NEWS), transurethral resection of the bladder tumor (TUR-bt), or the like is widely performed. In the minimally invasive treatment, in the case of performing treatment, for example, in order to mark a region to be operated as pretreatment, an operator such as a doctor performs resection by cauterization, marking treatment by thermal treatment, or the like on a characteristic region (a pathogenic region) having an affected part with respect to a biological tissue using a treatment tool of an energy device that emits energy such as high frequency, ultrasonic, or microwave. In addition, in the case of actual treatment as well, the operator performs treatment such as resection and coagulation of a biological tissue of a subject using an energy device or the like. Therefore, the endoscope system 1 illustrated in FIG. 1 is used when the subject is operated or processed using a treatment tool (not illustrated) such as an energy device capable of performing thermal treatment. Specifically, the endoscope system 1 illustrated in FIG. 1 is used for transurethral resection of bladder tumor (TUR-Bt), and is used when a treatment is performed on a tumor (bladder cancer) of the bladder or a pathogenic region.

The endoscope system 1 illustrated in FIG. 1 includes an insertion unit 2, a light source device 3, a light guide 4, an endoscope camera head 5 (endoscope imaging device), a first transmission cable 6, a display device 7, a second transmission cable 8, a control device 9, and a third transmission cable 10.

The insertion unit 2 is rigid or at least partially flexible and has an elongated shape. The insertion unit 2 is inserted into a subject such as a patient via a trocar. The insertion unit 2 is provided with an optical system such as a lens that forms an observation image therein.

The light source device 3 is connected to one end of the light guide 4, and supplies illumination light to irradiate the inside of the subject to one end of the light guide 4 under the control of the control device 9. The light source device 3 is realized by using one or more light sources of a light emitting diode (LED) light source, a xenon lamp, and a semiconductor laser element such as a laser diode (LD), a processor, which is a processing device having hardware such as a field programmable gate array (FPGA) and a central processing unit (CPU), and a memory, which is a temporary storage area used by the processor. Note that the light source device 3 and the control device 9 may be configured to communicate individually as illustrated in FIG. 1, or may be integrated with each other.

One end of the light guide 4 is detachably connected to the light source device 3, and the other end thereof is detachably connected to the insertion unit 2. The light guide 4 guides illumination light supplied from the light source device 3 from one end to the other end and supplies the illumination light to the insertion unit 2.

An eyepiece unit 21 of the insertion unit 2 is detachably connected to the endoscope camera head 5. Under the control of the control device 9, the endoscope camera head 5 generates an imaging signal (RAW data) by receiving an observation image formed by the insertion unit 2 and performing photoelectric conversion, and outputs the imaging signal to the control device 9 via the first transmission cable 6.

One end of the first transmission cable 6 is detachably connected to the control device 9 via a video connector 61, and the other end thereof is detachably connected to the endoscope camera head 5 via a camera head connector 62. The first transmission cable 6 transmits the imaging signal output from the endoscope camera head 5 to the control device 9, and transmits setting data, power, and the like output from the control device 9 to the endoscope camera head 5. Here, the setting data is a control signal, a synchronization signal, a clock signal, and the like for controlling the endoscope camera head 5.

Under the control of the control device 9, the display device 7 displays an observation image based on an imaging signal subjected to image processing in the control device 9 and various types of information regarding the endoscope system 1. The display device 7 is realized by using a display monitor such as liquid crystal or organic electro luminescence (EL).

One end of the second transmission cable 8 is detachably connected to the display device 7, and the other end thereof is detachably connected to the control device 9. The second transmission cable 8 transmits the imaging signal subjected to the image processing in the control device 9 to the display device 7.

The control device 9 is realized by using a processor, which is a processing device having hardware such as a graphics processing unit (GPU), an FPGA, or a CPU, and a memory, which is a temporary storage area used by the processor. The control device 9 integrally controls operations of the light source device 3, the endoscope camera head 5, and the display device 7 via each of the first transmission cable 6, the second transmission cable 8, and the third transmission cable 10 according to a program recorded in the memory. In addition, the control device 9 performs various types of image processing on the imaging signal input via the first transmission cable 6 and outputs the imaging signal to the second transmission cable 8.

One end of the third transmission cable 10 is detachably connected to the light source device 3, and the other end thereof is detachably connected to the control device 9. The third transmission cable 10 transmits the control data from the control device 9 to the light source device 3.

### Functional Configuration of Main Part of Endoscope System

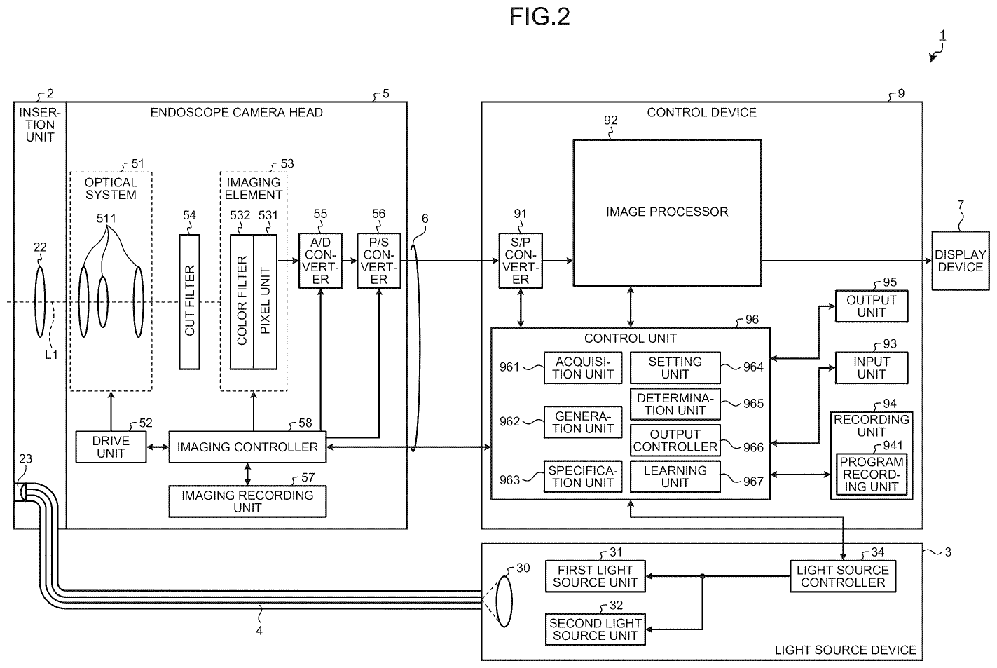

Next, a functional configuration of a main part of the above-described endoscope system 1 will be described. FIG. 2 is a block diagram illustrating a functional configuration of a main part of the endoscope system 1.

### Configuration of Insertion Unit

First, the configuration of the insertion unit 2 will be described. The insertion unit 2 includes an optical system 22 and an illumination optical system 23.

The optical system 22 forms a subject image by collecting light such as reflected light reflected from a subject, return light from the subject, excitation light from the subject, and fluorescence emitted from a thermally denatured region thermally denatured by thermal treatment of an energy device or the like. The optical system 22 is realized by using one or a plurality of lenses and the like.

The illumination optical system 23 irradiates the subject with illumination light supplied from the light guide 4. The illumination optical system 23 is realized by using one or a plurality of lenses or the like.

### Configuration of Light Source Device

Next, a configuration of the light source device 3 will be described. The light source device 3 includes a condenser lens 30, a first light source unit 31, a second light source unit 32, and a light source controller 33.

The condenser lens 30 condenses light emitted by each of the first light source unit 31 and the second light source unit 32 and emits the light to the light guide 4.

Under the control of the light source controller 33, the first light source unit 31 supplies white light as illumination light to the light guide 4 by emitting white light (normal light) which is visible light. The first light source unit 31 includes a collimator lens, a white LED lamp, a drive driver, and the like. Note that the first light source unit 31 may supply visible white light by simultaneously emitting light using a red LED lamp, a green LED lamp, and a blue LED lamp. Of course, the first light source unit 31 may be configured using a halogen lamp, a xenon lamp, or the like.

Under the control of the light source controller 33, the second light source unit 32 emits excitation light having a predetermined wavelength band to supply the excitation light to the light guide 4 as illumination light. Here, the excitation light has a wavelength band ranging from 400 nm to 430 nm (a center wavelength is 415 nm). The second light source unit 32 is realized by using a semiconductor laser such as a collimator lens or a violet laser diode (LD), a drive driver, and the like. In the first embodiment, the excitation light excites advanced glycation end-products generated by subjecting a biological tissue to thermal treatment by an energy device or the like. When the amino acid and the reducing sugar are heated, a saccharification reaction (Maillard reaction) occurs. The end product resulting from this Maillard reaction is generally called advanced glycation end-products (AGEs). As characteristics of the AGEs, it is known that a substance having fluorescence characteristics is included. That is, when the biological tissue is thermally treated with an energy device, the AGEs are generated by heating the amino acid and the reducing sugar in the biological tissue to cause the Maillard reaction. The AGEs generated by this heating can visualize the state of thermal treatment by fluorescence observation. Furthermore, AGEs are known to emit stronger fluorescence than autofluorescent substances originally present in biological tissues. That is, in the first embodiment, the thermally denatured region by the thermal treatment is visualized using the fluorescence characteristic of the AGEs generated in the biological tissue by the thermal treatment by the energy device or the like. Therefore, in the first embodiment, the biological tissue is irradiated with excitation light of blue light having a wavelength of about 415 nm for exciting the AGEs from the second light source unit 32. As a result, in the first embodiment, the fluorescence image (thermally denatured image) can be observed based on an imaging signal obtained by capturing the fluorescence (for example, green light having a wavelength ranging from 490 to 625 nm) emitted from the thermally denatured region generated from the AGEs.

The light source controller 33 is realized by using a processor having hardware such as an FPGA or a CPU, and a memory, which is a temporary storage area used by the processor. The light source controller 33 controls light emission timing, light emission time, and the like of each of the first light source unit 31 and the second light source unit 32 based on control data input from the control device 9.

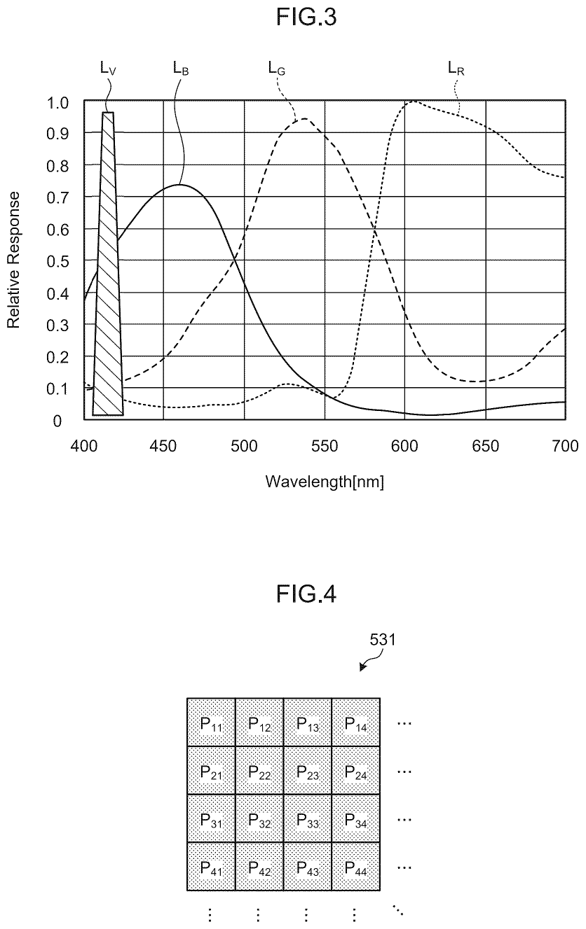

Here, wavelength characteristics of light emitted by the second light source unit 32 will be described. FIG. 3 is a diagram schematically illustrating wavelength characteristics of excitation light emitted by the second light source unit 32. In FIG. 3, the horizontal axis represents a wavelength (nm), and the vertical axis represents wavelength characteristics. In FIG. 3, a polygonal line L_{V} represents the wavelength characteristic of the excitation light emitted by the second light source unit 32. In FIG. 3, a curve L_{B} represents a blue wavelength band, a curve L_{G} represents a green wavelength band, and a curve L_{R} represents a red wavelength band.

As indicated by the polygonal line L_{V} in FIG. 3, the second light source unit 32 emits excitation light having a center wavelength (peak wavelength) of 415 nm and a wavelength band ranging from 400 nm to 430 nm.

### Configuration of Endoscope Camera Head

Referring back to FIG. 2, the description of the configuration of the endoscope system 1 will be continued.

Next, a configuration of the endoscope camera head 5 will be described. The endoscope camera head 5 includes an optical system 51, a drive unit 52, an imaging element 53, a cut filter 54, an A/D converter 55, a P/S converter 56, an imaging recording unit 57, and an imaging controller 58.

The optical system 51 forms a subject image collected by the optical system 22 of the insertion unit 2 on the light receiving surface of the imaging element 53. The optical system 51 can change the focal length and the focal position. The optical system 51 includes a plurality of lenses 511. The optical system 51 changes the focal length and the focal position by moving each of the plurality of lenses 511 on an optical axis L1 by the drive unit 52.

Under the control of the imaging controller 58, the drive unit 52 moves the plurality of lenses 511 of the optical system 51 along the optical axis L1. The drive unit 52 includes motors such as a stepping motor, a DC motor, and a voice coil motor, and a transmission mechanism such as a gear that transmits rotation of the motor to the optical system 51.

The imaging element 53 is implemented by using a charge coupled device (CCD) or complementary metal oxide semiconductor (CMOS) image sensor having a plurality of pixels arranged in a two-dimensional matrix. Under the control of the imaging controller 58, the imaging element 53 receives a subject image (light beam) that is formed by the optical system 51 and passes through the cut filter 54, performs photoelectric conversion, generates an imaging signal (RAW data), and outputs the imaging signal to the A/D converter 55. The imaging element 53 includes a pixel unit 531 and a color filter 532.

FIG. 4 is a diagram schematically illustrating a configuration of the pixel unit 531. As illustrated in FIG. 4, in the pixel unit 531, a plurality of pixels Pₙₘ (n=integer greater than or equal to 1, m=integer greater than or equal to 1) such as photodiodes that accumulate charges according to the amount of light are arranged in a two-dimensional matrix. Under the control of the imaging controller 58, the pixel unit 531 reads an image signal as image data from a pixel Pₙₘ in a reading region arbitrarily set as a reading target among the plurality of pixels Pₙₘ, and outputs the image signal to the A/D converter 55.

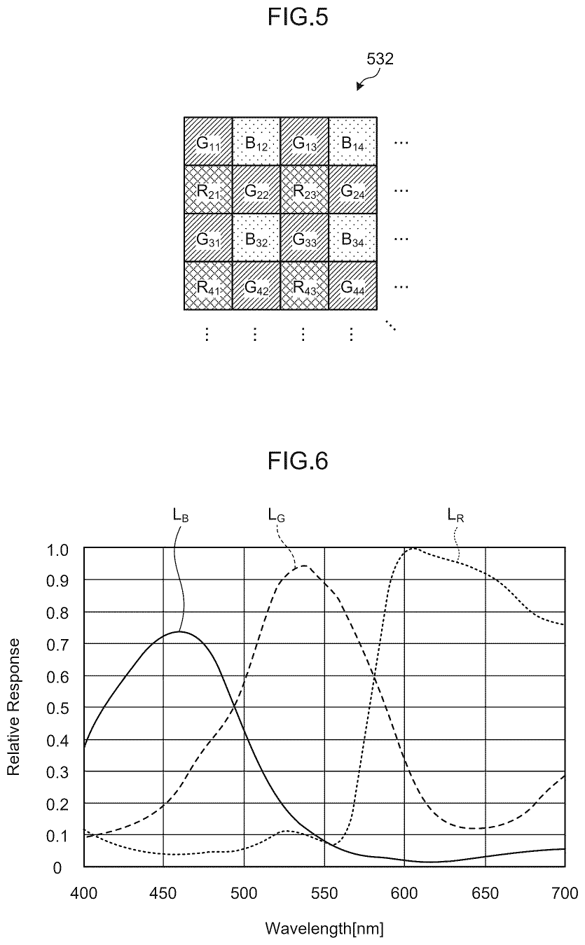

FIG. 5 is a diagram schematically illustrating a configuration of the color filter 532. As illustrated in FIG. 5, the color filter 532 includes a Bayer array having 2×2 as one unit. The color filter 532 includes a filter R that transmits light in a red wavelength band, two filters G that transmit light in a green wavelength band, and a filter B that transmits light in a blue wavelength band.

FIG. 6 is a diagram schematically illustrating sensitivity and a wavelength band of each filter. In FIG. 6, the horizontal axis represents a wavelength (nm), and the vertical axis represents transmission characteristics (sensitivity characteristics). In FIG. 6, a curve L_{B} represents the transmission characteristics of the filter B, a curve L_{G} represents the transmission characteristic of the filter G, and a curve L_{R} represents the transmission characteristic of the filter R.

As indicated by the curve L_{B} in FIG. 6, the filter B transmits light in a blue wavelength band. As indicated by the curve L_{G} in FIG. 6, the filter G transmits light in a green wavelength band. Further, as indicated by the curve L_{R} in FIG. 6, the filter R transmits light in a red wavelength band. Note that, in the following description, a pixel Pₙₘ in which the filter R is arranged on the light receiving surface will be described as an R pixel, a pixel Pₙₘ in which the filter G is arranged on the light receiving surface will be described as a G pixel, and a pixel Pₙₘ in which the filter B is arranged on the light receiving surface will be described as a B pixel.

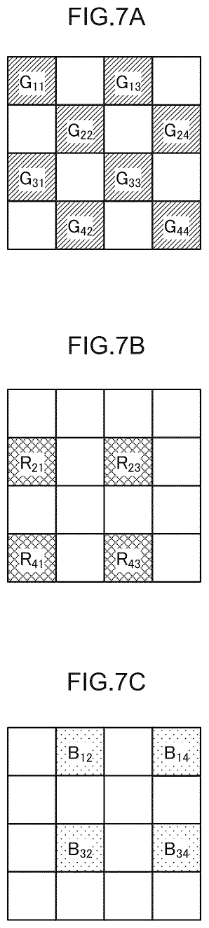

According to the imaging element 53 configured as described above, in a case where the subject image formed by the optical system 51 is received, a color signal (R signal, G signal, and B signal) of each of the R pixel, the G pixel, and the B pixel is generated (refer to FIGS. 7A to 7C).

Referring back to FIG. 2, the description of the configuration of the endoscope system 1 will be continued.

The cut filter 54 is disposed on the optical axis L1 between the optical system 51 and the imaging element 53. The cut filter 54 is provided on the light receiving surface side (incident surface side) of the G pixel provided with the filter G that transmits at least the green wavelength band of the color filter 532. The cut filter 54 shields light in a wavelength band of a short wavelength including a wavelength band of excitation light, and transmits a wavelength band on a longer wavelength side than the wavelength band of the excitation light.

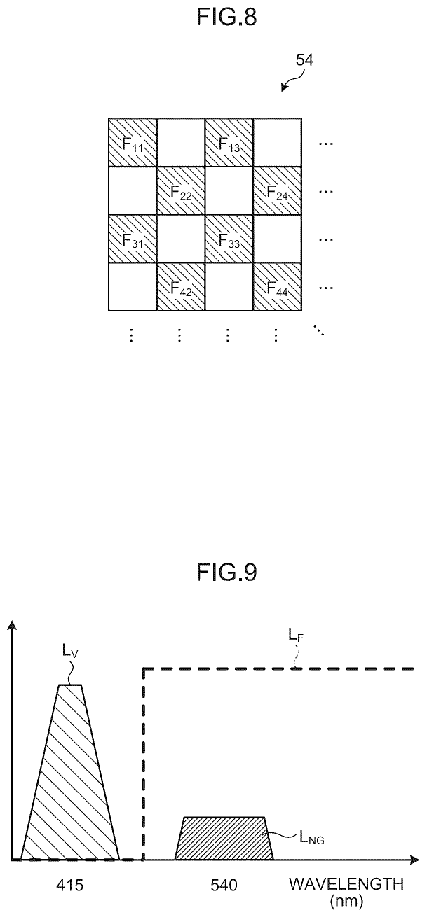

FIG. 8 is a diagram schematically illustrating a configuration of the cut filter 54. As illustrated in FIG. 8, a filter F₁₁ constituting the cut filter 54 is arranged at a position where the filter G₁₁ (refer to FIG. 5) is arranged, and is arranged on the light receiving surface side directly above the filter G₁₁.

FIG. 9 is a diagram schematically illustrating transmission characteristics of the cut filter 54. In FIG. 9, the horizontal axis represents a wavelength (nm), and the vertical axis represents transmission characteristics. In FIG. 9, a polygonal line LF represents a transmission characteristic of the cut filter 54, a polygonal line L_{NG} represents a wavelength characteristic of fluorescence, and a polygonal line L_{V} represents a wavelength characteristic of excitation light.

As illustrated in FIG. 9, the cut filter 54 shields a wavelength band of an excitation light and transmits a wavelength band on a long wavelength side from the wavelength band of the excitation light. Specifically, the cut filter 54 shields light in a wavelength band on a short wavelength side of 400 nm to less than 430 nm including the wavelength band of the excitation light, and transmits light in a wavelength band on a longer wavelength side than 400 nm to 430 nm including the excitation light.

Returning to FIG. 2, the description of the configuration of the endoscope camera head 5 will be continued.

Under the control of the imaging controller 58, the A/D converter 55 performs A/D conversion processing on an analog imaging signal input from the imaging element 53, and outputs the analog imaging signal to the P/S converter 56. The A/D converter 55 is implemented by using an A/D conversion circuit or the like.

Under the control of the imaging controller 58, the P/S converter 56 performs parallel/serial conversion on a digital imaging signal input from the A/D converter 55, and outputs the imaging signal subjected to the parallel/serial conversion to the control device 9 via the first transmission cable 6. The P/S converter 56 is implemented by using a P/S conversion circuit or the like. Note that, in the first embodiment, an E/O converter that converts an imaging signal into an optical signal may be provided instead of the P/S converter 56, and the imaging signal may be output to the control device 9 by the optical signal, or the imaging signal may be transmitted to the control device 9 by, for example, wireless communication such as Wireless Fidelity (Wi-Fi)™.

The imaging recording unit 57 records various types of information (for example, pixel information of the imaging element 53 and characteristics of the cut filter 54) regarding the endoscope camera head 5. Furthermore, the imaging recording unit 57 records various setting data and control parameters transmitted from the control device 9 via the first transmission cable 6. The imaging recording unit 57 is configured using a nonvolatile memory or a volatile memory.

The imaging controller 58 controls the operation of each of the drive unit 52, the imaging element 53, the A/D converter 55, and the P/S converter 56 based on the setting data received from the control device 9 via the first transmission cable 6. The imaging controller 58 is realized by using a timing generator (TG), a processor having hardware such as an application specific integrated circuit (ASIC) or a CPU, and a memory, which is a temporary storage area used by the processor.

### Configuration of Control Device

Next, a configuration of the control device 9 will be described.

The control device 9 includes an S/P converter 91, an image processor 92, an input unit 93, a recording unit 94, and a control unit 96.

Under the control of the control unit 96, the S/P converter 91 performs serial/parallel conversion on image data received from the endoscope camera head 5 via the first transmission cable 6 and outputs the image data to the image processor 92. Note that, in a case where the endoscope camera head 5 outputs an imaging signal as an optical signal, an O/E converter that converts an optical signal into an electric signal may be provided instead of the S/P converter 91. Furthermore, in a case where the endoscope camera head 5 transmits an imaging signal by wireless communication, a communication module capable of receiving a wireless signal may be provided instead of the S/P converter 91.

Under the control of the control unit 96, the image processor 92 performs predetermined image processing on an imaging signal of parallel data input from the S/P converter 91 and outputs the imaging signal to the display device 7. Here, the predetermined image processing is demosaic processing, white balance processing, gain adjustment processing, y correction processing, format conversion processing, and the like. The image processor 92 is implemented by using a processor which is a processing device having hardware such as a GPU or an FPGA and a memory which is a temporary storage area used by the processor.

The input unit 93 receives inputs of various operations related to the endoscope system 1 and outputs the received operations to the control unit 96. The input unit 93 includes a mouse, a foot switch, a keyboard, a button, a switch, a touch panel, and the like.

The recording unit 94 is implemented by using a recording medium such as a volatile memory, a nonvolatile memory, a solid state drive (SSD), a hard disk drive (HDD), or a memory card. The recording unit 94 records data including various parameters and the like necessary for the operation of the endoscope system 1. Furthermore, the recording unit 94 includes a program recording unit 941 that records various programs for operating the endoscope system 1.

An output unit 95 outputs various types of information under the control of the control unit 96. The output unit 95 is configured using, for example, a speaker, a display panel, and the like.

The control unit 96 is realized by using a processor having hardware such as an FPGA or a CPU, and a memory, which is a temporary storage area used by the processor. The control unit 96 integrally controls each of the units constituting the endoscope system 1. Specifically, the control unit 96 reads and executes a program recorded in the program recording unit 941 in a work area of a memory, and controls each component and the like through execution of the program by the processor, so that hardware and software cooperate with each other to realize a functional module matching a predetermined purpose. Specifically, the control unit 96 includes an acquisition unit 961, a generation unit 962, a specification unit 963, a setting unit 964, a determination unit 965, an output controller 966, and a learning unit 967.

The acquisition unit 961 acquires an imaging signal generated by capturing an image by the endoscope camera head 5 via the insertion unit 2.

The generation unit 962 generates a white light image based on the imaging signal acquired by the acquisition unit 961. The generation unit 962 performs demosaic processing, white balance processing, gain adjustment processing, Y correction processing, and the like on the imaging signal acquired by the acquisition unit 961 to generate the white light image.

The specification unit 963 specifies a specific region from the white light image generated by the generation unit 962. The specification unit 963 specifies a specific region A1 from a white light image P1 using a well-known technique such as pattern matching.

The setting unit 964 sets a circle having a predetermined radius centered on the specific region A1 specified by the specification unit 963 as a detection range.

The determination unit 965 determines whether a fluorescence region having a fluorescence amount equal to or more than a fluorescence amount recognized as thermal denaturation is generated in the fluorescence image.

The output controller 966 outputs information indicating that a light emitting region is within a detection range R1 to the display device 7.

The learning unit 967 may generate a learned model by performing machine learning using teacher data in which a fluorescence image obtained by capturing fluorescence generated by irradiating a biological tissue with excitation light and a white light image obtained by capturing an image generated by irradiating the biological tissue with white light are used as input data and information indicating a relationship between a thermally denatured region extracted from the fluorescence image and a position (coordinates) of an organ or an entrance/exit of the organ in a specific region specified from the white light image is used as output data.

Here, the learned model includes a neural network in which each layer has one or a plurality of nodes. In addition, the type of machine learning is not particularly limited, and for example, it is sufficient that training data and learning data in which fluorescence images of a plurality of subjects and white light images of a plurality of subjects are associated with positions of an organ or an entrance/exit of the organ in a specific region specified from the plurality of fluorescence images and the plurality of white light images are prepared, and the training data and the learning data are input to a calculation model based on a multilayer neural network for learning.

Furthermore, as a machine learning method, for example, a method based on a deep neural network (DNN) of a multilayer neural network such as a convolutional neural network (CNN) or a 3D-CNN is used.

Furthermore, as a machine learning method, a method based on a recurrent neural network (RNN), a long short-term memory unit (LSTM) obtained by extending the RNN, or the like may be used. Note that a learning unit of a learning device different from the control device 9 may execute these functions to generate a learned model. Of course, the function of the learning unit 967 may be provided in the image processor 92.

### Processing of Control Device

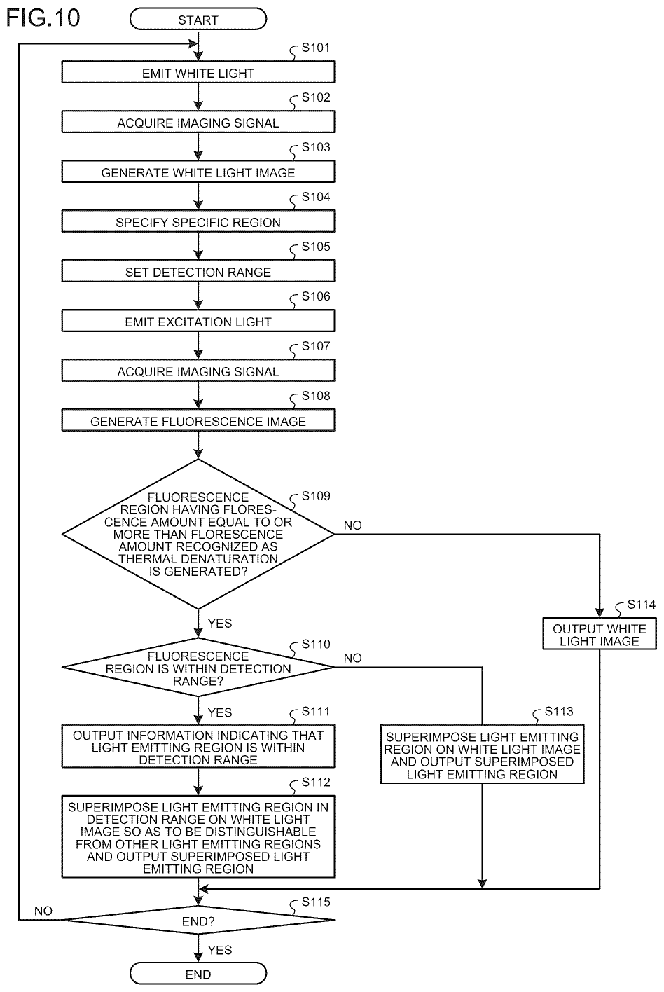

Next, processing executed by the control device 9 will be described. FIG. 10 is a flowchart illustrating an outline of processing executed by the control device 9.

First, as illustrated in FIG. 10, the control unit 96 causes the first light source unit 31 of the light source device 3 to emit light and supplies white light to the insertion unit 2 to irradiate the biological tissue with white light (Step S101).

Subsequently, the acquisition unit 961 acquires an imaging signal generated by capturing an image using the endoscope camera head 5 via the insertion unit 2 (Step S102).

Thereafter, the generation unit 962 generates a white light image based on the imaging signal acquired by the acquisition unit 961 (Step S103). Specifically, the generation unit 962 performs demosaic processing, white balance processing, gain adjustment processing, Y correction processing, and the like on the imaging signal acquired by the acquisition unit 961 to generate the white light image. Note that the generation unit 962 generates the white light image based on the imaging signal acquired by the acquisition unit 961, but is not limited thereto, and may acquire the white light image generated by the image processor 92.

Subsequently, the specification unit 963 specifies a specific region from the white light image generated by the generation unit 962 (Step S104).

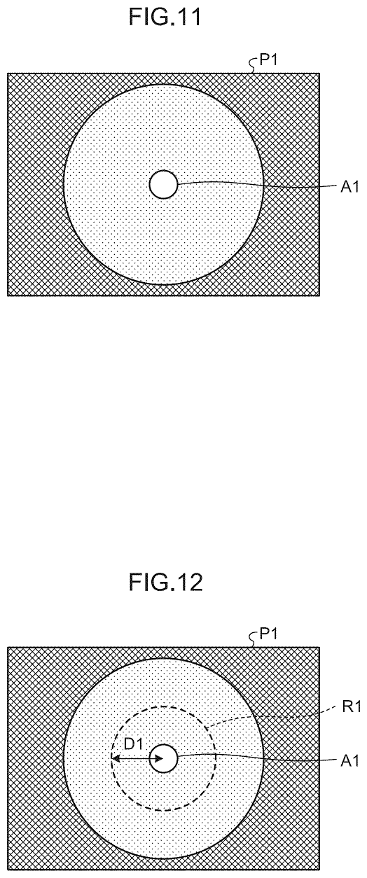

FIG. 11 is a diagram schematically illustrating a specific region in the white light image specified by the specification unit 963. As illustrated in FIG. 11, the specification unit 963 specifies a specific region A1 from a white light image P1 using a well-known technique such as pattern matching. Here, the specific region is an entrance/exit of an organ such as a ureteral orifice, a cardia, a pylorus, a pancreatic duct, and a bile duct, and a predetermined organ. Specifically, the specification unit 963 specifies the specific region A1 by performing known pattern matching on the white light image P1 using a pattern for each organ set in advance.

In the first embodiment, a case of the ureteral orifice as the specific region A1 of the white light image P1 will be described. Furthermore, the specification unit 963 may specify the specific region A1 from the white light image P1 using a learning result (a learned model) obtained by performing machine learning such as deep learning on teacher data in which the plurality of white light images P1 are associated with a region (position information) of the organ included in each of the plurality of white light images P1 and annotation information to which the type of the organ is added. In this case, the specification unit 963 may have input data as the white light image P1, and may output, as the specific region A1, output data indicating the type of an organ and the region of the organ (position information of the organ) in the white light image P1.

Furthermore, the specification unit 963 may specify the specific region A1 in the white light image P1 by performing any one of edge detection processing, blob analysis processing, and binarization processing on a pixel value of each pixel constituting the white light image P1.

Referring back to FIG. 10, in Step S105, the setting unit 964 sets, as a detection range, a circle having a predetermined radius centered on the specific region A1 specified by the specification unit 963.

FIG. 12 is a diagram schematically illustrating a detection range set by the setting unit 964. As illustrated in FIG. 12, the setting unit 964 sets a detection range R1 for detecting a thermally denatured region in the white light image P1 (thermally denatured image) based on the specific region specified by the specification unit 963. Specifically, the setting unit 964 sets, as a detection range, a circle having a predetermined radius D1 centered on the specific region A1 specified by the specification unit 963. In this case, the setting unit 964 sets the length of the radius D1 based on at least one of the type and the size of the organ in the specific region A1 specified by the specification unit 963.

Note that, in FIG. 12, the setting unit 964 sets the shape of the detection range as a circle, but the shape is not limited thereto and can be appropriately changed, and may be, for example, a polygon, a quadrangle, or a pentagon. In addition, the setting unit 964 may set a circle centered on the specific region A1 as the detection range R1 by a radius designated by a user input from the input unit 93.

Referring back to FIG. 10, in Step S106, the control unit 96 causes the second light source unit 32 of the light source device 3 to emit light and supplies excitation light to the insertion unit 2, thereby irradiating a biological tissue with the excitation light.

Subsequently, the acquisition unit 961 acquires an imaging signal generated by capturing an image using the endoscope camera head 5 via the insertion unit 2 (Step S107).

Thereafter, the generation unit 962 generates a fluorescence image as a thermally denatured image including at least a thermally denatured region based on the imaging signal acquired by the acquisition unit 961 (Step S108).

The determination unit 965 determines whether a fluorescence region having a fluorescence amount equal to or more than a fluorescence amount recognized as thermal denaturation is generated in the fluorescence image (Step S109). Specifically, the determination unit 965 determines whether a pixel value (luminance value) is greater than or equal to the amount of fluorescence (luminance value) recognized as thermal denaturation for each pixel constituting the fluorescence image or for each predetermined number of pixels, determines that the fluorescence region is generated in the fluorescence image when the number of pixels, the pixel value (luminance value) of which is greater than or equal to the amount of fluorescence (luminance value) recognized as thermal denaturation, is greater than or equal to a predetermined number, and determines that the fluorescence region is not generated in the fluorescence image when the number of pixels, the pixel value (luminance value) of which is greater than or equal to the amount of fluorescence (luminance value) recognized as thermal denaturation, is less than the predetermined number. When the determination unit 965 determines that the fluorescence region having the fluorescence amount equal to or more than the fluorescence amount recognized as thermal denaturation is generated in the fluorescence image (Step S109: Yes), the control device 9 proceeds to Step S110 described later. On the other hand, when the determination unit 965 determines that the fluorescence region having the fluorescence amount equal to or more than the fluorescence amount recognized as thermal denaturation is not generated in the fluorescence image (Step S109: No), the control device 9 proceeds to Step S115 described later.

In Step S110, the determination unit 965 determines whether the fluorescence region is within the detection range R1 set by the setting unit 964. When the determination unit 965 determines that the fluorescence region is within the detection range R1 set by the setting unit 964 (Step S110: Yes), the control device 9 proceeds to Step S111 described later. On the other hand, when the determination unit 965 determines that the fluorescence region is not within the detection range R1 set by the setting unit 964 (Step S110: No), the control device 9 proceeds to Step S113 described later.

In Step S111, the output controller 966 outputs information indicating that a light emitting region is within the detection range R1 to the display device 7. In this case, the output controller 966 outputs text data to the display device 7 as information indicating that the light emitting region is within the detection range R1. Note that the output controller 966 may output an output signal for causing the display device 7 to output sound as information indicating that the light emitting region is within the detection range R1.

Subsequently, the output controller 966 causes the image processor 92 to superimpose the light emitting region in the detection range R1 on the white light image P1 so as to be distinguishable from other light emitting regions, and then outputs the light emitting region superimposed on the white light image to the display device 7 (Step S112).

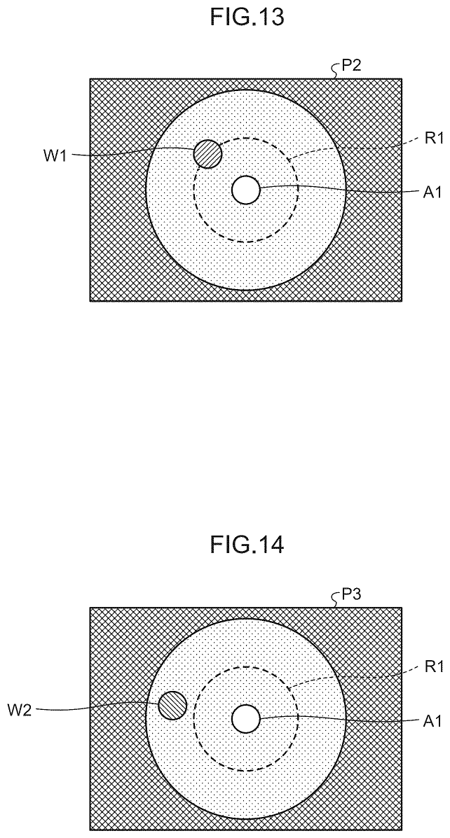

FIG. 13 is a diagram illustrating an example of a display image displayed by the display device 7. As illustrated in FIG. 13, the output controller 966 outputs, to the display device 7, a display image P2 obtained in such a manner that a light emitting region W1 in the detection range R1 is emphasized so as to be distinguishable from other light emitting regions and then is superimposed on the white light image P1 by the image processor 92. Specifically, the output controller 966 outputs, to the display device 7, the display image P2 obtained in such a manner that the color of the light emitting region W1, which is the thermally denatured region in the detection range R1, is converted into a color to be enhanced, for example, any one of red, blue, and black, and then is superimposed on the white light image P1 by the image processor 92. In this case, the output controller 966 may change the color to be enhanced stepwise according to the light emission intensity of the light emitting region W1, or may change saturation, brightness, or the like and may superimpose the color on the white light image P1. Of course, the output controller 966 may output, to the display device 7, the display image P2 superimposed on the white light image P1 by enabling identification of only a part of the light emitting region W1, for example, the outline of the light emitting region W1, or may output, to the display device 7, the display image P2 superimposed on the white light image P1 by enabling identification with a color or the like designated in advance by the user. As a result, the user can intuitively grasp that the light emitting region W1 is included in the detection range R1. After Step S112, the control device 9 proceeds to Step S115 described later.

In Step S113, the output controller 966 causes the image processor 92 to superimpose the light emitting region on the white light image P1, and then outputs the light emitting region superimposed on the white light image to the display device 7.

FIG. 14 is a diagram illustrating an example of a display image displayed by the display device 7. As illustrated in FIG. 14, the output controller 966 outputs, to the display device 7, a display image P3 obtained by causing the image processor 92 to superimpose the light emitting region W2 on the white light image P1. As a result, the user can intuitively grasp the position of the light emitting region W2. After Step S113, the control device 9 proceeds to Step S115 described later.

In Step S114, the output controller 966 causes the image processor 92 to output the white light image P1 to the display device 7. After Step S114, the control device 9 proceeds to Step S115 described later.

In Step S115, the determination unit 965 determines whether an end signal for ending the observation of the subject by the endoscope system 1 is input from the input unit 93. When the determination unit 965 determines that the end signal for ending the observation of the subject by the endoscope system 1 is input from the input unit 93 (Step S115: Yes), the control device 9 ends the present processing. On the other hand, when the determination unit 965 determines that the end signal for ending the observation of the subject by the endoscope system 1 is not input from the input unit 93 (Step S115: No), the control device 9 returns to Step S101 described above.

According to the first embodiment described above, the output controller 966 outputs thermal denaturation information to the display device 7 or the output unit 95 based on the detection range set by the setting unit 964 and the thermally denatured region in the white light image, which is the thermally denatured image acquired by the acquisition unit 961. As a result, the user can confirm the state of thermal denaturation in the specific region.

According to the first embodiment, when the determination unit 965 determines that the fluorescence region, which is the thermally denatured region, is included in the detection range, the output controller 966 outputs the thermal denaturation information to the output unit 95 or the display device 7. As a result, the user can grasp that the thermally denatured region is included in the specific region.

Furthermore, according to the first embodiment, the output controller 966 outputs, to the display device 7, the display image P2 obtained in such a manner that the light emitting region W1 in the detection range R1 is emphasized so as to be distinguishable from other light emitting regions and then is superimposed on the white light image P1 by the image processor 92. As a result, the user can intuitively grasp the light emitting region W1 in the detection range R1.

Furthermore, according to the first embodiment, since the determination unit 965 determines whether a thermally denatured region is generated by thermal treatment based on a signal value of each pixel in the fluorescence image, it is possible to make a determination separately from fluorescence of an autofluorescent substance originally present in a biological tissue.

Furthermore, according to the first embodiment, since the specification unit 963 specifies a specific region including an organ and the like based on feature data in a white light image, the specific region can be easily specified from the white light image.

Furthermore, according to the first embodiment, since the setting unit 964 sets the detection range based on the type of the specific region, it is possible to automatically set the optimum detection range according to the specific region.

Note that, in the first embodiment, the specification unit 963 desires to specify the specific region based on the feature data in the white light image, but the disclosure is not limited thereto, and for example, the specific region in the white light image may be specified based on an instruction signal input by a user operating the input unit 93.

Furthermore, in the first embodiment, the setting unit 964 sets the detection range based on the type of the specific region, but the disclosure is not limited thereto, and for example, the detection range may be set based on an instruction signal input by a user operating the input unit 93. As a result, a detection range of a range desired by a user can be set.

Furthermore, in the first embodiment, the setting unit 964 may set the detection range based on a type of the specific region and a distance from a distal end portion of the insertion unit 2 to a specific region. In this case, the setting unit 964 calculates the distance from the distal end portion of the insertion unit 2 to the specific region based on one white light image or two temporally continuous white light images, and sets the detection range based on the calculation result and the type of the specific region. As a result, it is possible to set a detection range suitable for the current operation. Note that, although the setting unit 964 calculates the distance from the distal end portion to the specific region using the white light image, the disclosure is not limited thereto, and for example, a distance sensor, a distance measuring sensor, or the like may be provided at the distal end portion of the insertion unit 2, and the distance from the distal end portion to the specific region may be calculated based on the detection results of these sensors.

In the first embodiment, the recording unit 94 may be caused to record distance information in which the type of the specific region and the distance of the detection range are associated with each other, the setting unit 964 may acquire the distance information from the recording unit 94, and the detection range may be set based on the distance information and the type of the specific region specified by the specification unit 963.

Furthermore, in the first embodiment, the output controller 966 outputs, to the display device 7, the display image P2 obtained in such a manner that the light emitting region W1 in the detection range R1 is emphasized so as to be distinguishable from other light emitting regions and then is superimposed on the white light image P1 by the image processor 92. However, the disclosure is not limited thereto, and the detection range R1 set by the setting unit 964 may be superimposed on the fluorescence image W1, which is a thermally denatured image, and may be output to the display device 7. As a result, the user can intuitively grasp the detection range R1 in the fluorescence image W1.

Furthermore, in the first embodiment, the learning unit 967 is provided in the control device 9, but the disclosure is not limited thereto, and the learning unit 967 that generates a learned model may be provided in a device different from the control device 9, for example, a learning device or a server connectable via a network.

### Second Embodiment

Next, a second embodiment will be described. In the above-described first embodiment, the control unit 96 of the control device 9 determines whether there is a fluorescence region within a detection range, and outputs the determination result to the display device 7. However, in the second embodiment, a medical device that determines whether there is a fluorescence region within the detection range is separately provided. Hereinafter, a configuration of an endoscope system according to the second embodiment will be described. Note that the same components as those of the endoscope system 1 according to the first embodiment described above are denoted by the same reference numerals, and a detailed description thereof will be omitted.

### Configuration of Endoscope System

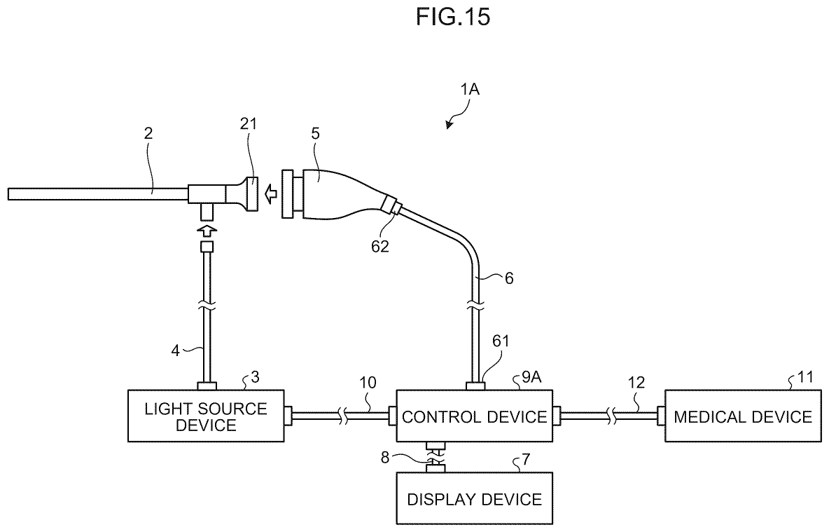

FIG. 15 is a diagram illustrating a schematic configuration of the endoscope system according to the second embodiment. An endoscope system 1A illustrated in FIG. 15 includes a control device 9A instead of the control device 9 of the endoscope system 1 according to the first embodiment described above. Furthermore, the endoscope system 1A further includes a medical device 11 and a fourth transmission cable 12 in addition to the configuration of the endoscope system 1 according to the first embodiment described above.

The control device 9A is realized by using a processor, which is a processing device having hardware such as a GPU, an FPGA, or a CPU, and a memory, which is a temporary storage area used by the processor. The control device 9A integrally controls operations of the light source device 3, the endoscope camera head 5, and the display device 7, and the medical device 11 via each of the first transmission cable 6, the second transmission cable 8, and the third transmission cable 10, and the fourth transmission cable 12 according to a program recorded in the memory. The control device 9A omits the functions of the acquisition unit 961, the generation unit 962, the specification unit 963, the setting unit 964, the determination unit 965, the output controller 966, and the learning unit 967 from the control unit 96 according to the first embodiment described above.

The medical device 11 is realized by using a processor, which is a processing device having hardware such as a GPU, an FPGA, or a CPU, and a memory, which is a temporary storage area used by the processor. The medical device 11 acquires various types of information from the control device 9A via the fourth transmission cable 12, and outputs the acquired various types of information to the control device 9A. Note that a detailed functional configuration of the medical device 11 will be described later.

One end of the fourth transmission cable 12 is detachably connected to the control device 9A, and the other end thereof is detachably connected to the medical device 11. The fourth transmission cable 12 transmits various types of information from the control device 9A to the medical device 11, and transmits various types of information from the medical device 11 to the control device 9A.

### Functional Configuration of Medical Device

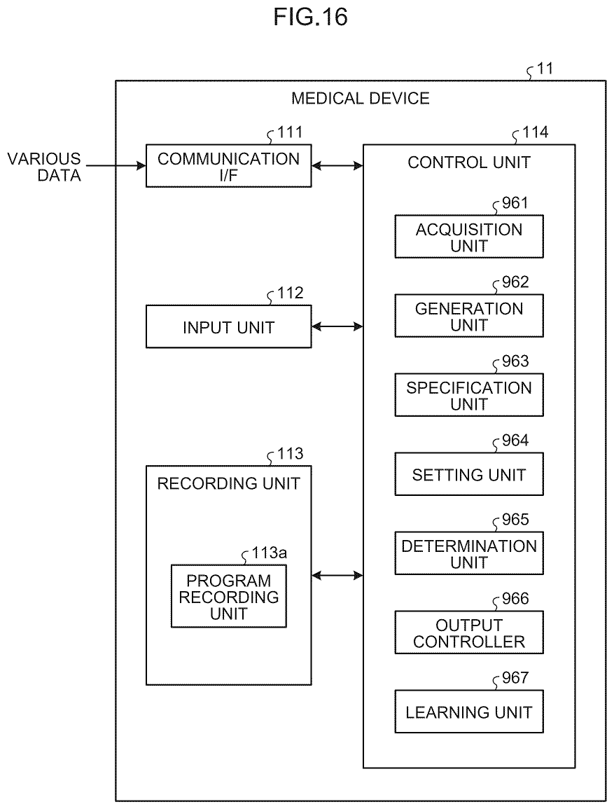

FIG. 16 is a block diagram illustrating a functional configuration of the medical device 11. The medical device 11 illustrated in FIG. 16 includes a communication I/F 111, an input unit 112, a recording unit 113, and a control unit 114.

The communication I/F 111 is an interface for communicating with the control device 9A via the fourth transmission cable 12. The communication I/F 111 receives various types of information from the control device 9A according to a predetermined communication standard, and outputs the received various types of information to the control unit 114.

The input unit 112 receives inputs of various operations related to the endoscope system 1A and outputs the received operations to the control unit 114. The input unit 112 includes a mouse, a foot switch, a keyboard, a button, a switch, a touch panel, and the like.

The recording unit 113 is realized using a recording medium such as a volatile memory, a nonvolatile memory, an SSD, an HDD, or a memory card. The recording unit 113 records therein data including various parameters and the like necessary for the operation of the medical device 11. Furthermore, the recording unit 113 includes a program recording unit 113a that records various programs for operating the medical device 11.

The control unit 114 is realized by using a processor having hardware such as an FPGA or a CPU, and a memory, which is a temporary storage area used by the processor. The control unit 114 integrally controls each unit constituting the medical device 11. The control unit 114 has the same function as the control unit 96 according to the first embodiment described above. Specifically, the control unit 114 includes a generation unit 962, a specification unit 963, a setting unit 964, a determination unit 965, an output controller 966, and a learning unit 967.

The medical device 11 configured as described above executes processing similar to that of the control device 9 according to the first embodiment described above, and outputs the processing result to the control device 9A. In this case, the control device 9A causes the image processor 92 to output a display image according to the presence or absence of the light emitting region in the detection range R1 of the white light image generated by the image processor 92 based on the processing result of the medical device 11, and causes the display device 7 to display the display image.

According to the second embodiment described above, the same effects as those of the first embodiment described above are obtained, and the user can confirm the state of thermal denaturation in the specific region.

### OTHER EMBODIMENTS

Various embodiments can be formed by appropriately combining a plurality of components disclosed in the endoscope system according to the first and second embodiments of the present disclosure described above. For example, some components may be deleted from all the components described in the endoscope system according to the embodiment of the present disclosure described above. Furthermore, the components described in the endoscope system according to the embodiment of the present disclosure described above may be appropriately combined with each other.

In addition, the endoscope systems according to the first and second embodiments of the present disclosure are connected to each other by wire, but may be connected to each other wirelessly via a network.

Furthermore, in the first and second embodiments of the present disclosure, the function of the control unit included in the endoscope system, and the functional modules of the generation unit 962, the specification unit 963, the setting unit 964, the determination unit 965, and the output controller 966 may be provided in a server or the like connectable via a network. Of course, a server may be provided for each functional module.

In addition, in the first and second embodiments of the present disclosure, an example of being used for transurethral bladder tumor resection has been described, but the disclosure is not limited thereto, and can be applied to various treatments of resection of a lesion by, for example, an energy device or the like.

Furthermore, in the endoscope systems according to the first and second embodiments of the present disclosure, the above-described “unit” can be replaced with “means”, “circuit”, or the like. For example, the control unit can be replaced with a control means or a control circuit.

Note that, in the description of the flowcharts in the present specification, the context of processing between steps is clearly indicated using expressions such as “first”, “thereafter”, and “subsequently”, but the order of processing necessary for implementing the embodiments is not uniquely determined by such expressions. That is, the order of processing in the flowcharts described in the present specification can be changed within a range without inconsistency.

According to the present disclosure, there is an effect that a state of thermal denaturation according to a specific region can be confirmed.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical device comprising a processor comprising hardware, the processor being configured to:
acquire a thermally denatured image including at least a thermally denatured region;
specify a specific region included in the thermally denatured image;
set, based on the specific region, a detection range serving as a target range for detecting the thermally denatured region in the thermally denatured image;
determine whether the thermally denatured region is included in the detection range; and
output thermal denaturation information when the thermally denatured region is included in the detection range.

2. The medical device according to claim 1, wherein
the processor is further configured to output, when the thermally denatured region is included in the detection range, the thermally denatured region in a more emphasized manner than when the thermally denatured region is not included in the detection range.

3. The medical device according to claim 1, wherein
the processor is further configured to output the thermally denatured region included in the detection range and the thermally denatured region not included in the detection range in different forms so as to be distinguishable from each other.

4. The medical device according to claim 1, wherein the processor is further configured to:
acquire an imaging signal generated by an imaging device; and
generate the thermally denatured image based on the imaging signal.

5. The medical device according to claim 4, wherein
the thermally denatured image is a fluorescence image.

6. The medical device according to claim 5, wherein
the processor is further configured to generate, when a biological tissue is irradiated with excitation light, the fluorescence image based on the imaging signal generated by capturing fluorescence emitted from the thermally denatured region using the imaging device.

7. The medical device according to claim 6, wherein
the fluorescence is emitted from an advanced glycation end-product generated by subjecting the biological tissue to a thermal treatment.

8. The medical device according to claim 5, wherein
the processor is further configured to determine whether the thermally denatured region is generated based on a signal value of each pixel in the fluorescence image.

9. The medical device according to claim 1, wherein the processor is further configured to:
acquire an imaging signal generated by an imaging device; and
generate a white light image based on the imaging signal.

10. The medical device according to claim 9, wherein
the processor is further configured to specify the specific region based on the white light image.

11. The medical device according to claim 10, wherein
the processor is further configured to specify the specific region based on feature data in the white light image.

12. The medical device according to claim 10, wherein
the processor is further configured to specify the specific region based on an instruction signal input from an outside.

13. The medical device according to claim 1, wherein
the processor is further configured to superimpose the detection range on the thermally denatured image and to output the thermally denatured image on which the detection range is superimposed to a display device.

14. The medical device according to claim 13, wherein
the processor is further configured to set the detection range based on a type of the specific region.

15. The medical device according to claim 13, wherein
the processor is further configured to set the detection range based on an instruction signal input from an outside.

16. The medical device according to claim 13, wherein
the processor is further configured to set the detection range based on a type of the specific region and a distance from a distal end portion of an imaging device to the specific region.

17. The medical device according to claim 16, wherein
the processor is further configured to:
acquire distance information from a memory configured to record the distance information in which the type of the specific region and a distance of the detection range are associated with each other; and
set the detection range based on the type of the specific region and the distance information.

18. The medical device according to claim 10, wherein
the processor is further configured to superimpose the thermally denatured region on the white light image and to output the while light image on which the thermally denatured region is superimposed to a display device.

19. The medical device according to claim 18, wherein
the processor further is configured to superimpose the thermally denatured region within the detection range and the thermally denatured region outside the detection range on the white light image in different forms and to output the white light image on which the thermally denatured region within the detection range and the thermally denatured region outside the detection range are superimposed to the display device.

20. The medical device according to claim 10, wherein
the specific region is an organ or an entrance or an exit of the organ.

21. A learning device comprising a processor comprising hardware, the processor being configured to generate a learned model by performing machine learning using teacher data in which a fluorescence image obtained by capturing fluorescence generated by irradiating a biological tissue with excitation light and a white light image obtained by capturing an image generated by irradiating the biological tissue with white light are used as input data, and information indicating a relationship between a thermally denatured region extracted from the fluorescence image and a specific region specified from the white light image is used as output data.

22. A medical system comprising:
a light source device including a light source configured to emit excitation light for exciting an advanced glycation end-product generated by subjecting a biological tissue to a thermal treatment;
an imaging device including an imaging element configured to generate an imaging signal by capturing fluorescence emitted by the excitation light; and
a medical device comprising a processor comprising hardware, the processor being configured to:
acquire a thermally denatured image including at least a thermally denatured region;
specify a specific region included in the thermally denatured image;
set, based on the specific region, a detection range serving as a target range for detecting the thermally denatured region in the thermally denatured image;
determine whether the thermally denatured region is included in the detection range; and
output thermal denaturation information when the thermally denatured region is included in the detection range.

23. An operation method of a medical device comprising a processor, the operation method causing the processor to execute:
acquiring a thermally denatured image including at least a thermally denatured region;
specifying a specific region included in the thermally denatured image;
setting, based on the specific region, a detection range serving as a target range for detecting the thermally denatured region in the thermally denatured image;
determining whether the thermally denatured region is included in the detection range; and
outputting thermal denaturation information when the thermally denatured region is included in the detection range.

24. A non-transitory computer-readable recording medium with an executable program stored thereon, the program causing a processor of a medical device driven to execute:
acquiring a thermally denatured image including at least a thermally denatured region;
specifying a specific region included in the thermally denatured image;
setting, based on the specific region, a detection range serving as a target range for detecting the thermally denatured region in the thermally denatured image;
determining whether the thermally denatured region is included in the detection range; and
outputting thermal denaturation information when the thermally denatured region is included in the detection range.
